(19) United States
(12) Patent Application Publication
(10) Pub. No.: US 2021/0005281 A1
(43) Pub. Date: Jan. 7, 2021

(54) GENE MUTATION ASSESSMENT DEVICE, ASSESSMENT METHOD, PROGRAM, AND STORAGE MEDIUM

(71) Applicants: NEC Corporation, Tokyo (JP); OSAKA UNIVERSITY, Suita-shi, Osaka (JP)

(72) Inventors: Masataka KIKUCHI, Suita-shi, Osaka (JP); Akihiro NAKAYA, Suita-shi, Osaka (JP)

(73) Assignees: NEC Corporation, Tokyo (JP); OSAKA UNIVERSITY, Suita-shi, Osaka (JP)

(21) Appl. No.: 16/976,808

(22) PCT Filed: Sep. 28, 2018
(86) PCT No.: PCT/JP2018/036376

§ 371 Date: Aug. 31, 2020

(30) Foreign Application Priority Data
Mar. 19, 2018 (JP) 2018-051268

(51) Int. Cl. G16B 20/50; G16H 50/70; G16B 50/00

(52) U.S. Cl. CPC G16B 20/50; G16B 50/00; G16H 50/70

(57) ABSTRACT

The present invention provides a new gene mutation assessment system which makes it possible, even when a gene mutation has considered to be apparently not associated with a trait from mutation information at a single position, to pick the gene mutation as a gene mutation candidate showing an association with the trait. The gene mutation assessment device (10) of the present invention includes a communication unit (19) that is capable of communicating a database DB, an assessment target mutation information acquisition unit (11) that acquires mutation information of a common gene mutation in a sample group showing a common trait as mutation information of an assessment target mutation, a score assignment unit (12) that assigns a first score showing an association with a trait of the DB information to the assessment target mutation based on the DB information, a score determination unit (13) that compares the first score with an association threshold and determines the assessment target mutation as a re-scoring target when the first score is less than the association threshold, a region mutation information acquisition unit (14) that acquires, as region mutation information, a gene mutation in an associated region with respect to a re-scoring target assessment target mutation based on the DB information, a score re-assignment unit (15) that assigns a second score weighted to the first score to the re-scoring target assessment target mutation based on the region mutation information, and an assessment score determination unit (16) that determines the second score as an assessment score of the re-scoring target assessment target mutation.

### TECHNICAL FIELD

The present invention relates to a gene mutation assessment device, an assessment method, a program, and a storage medium.

### BACKGROUND ART

Since gene mutations affect various traits, it is important to extract gene mutation and analyze what kind of trait is associated with the gene mutation. While examples of the trait generally include diseases and the responsiveness to a drug, in recent years, attention has been paid not only to these traits but also to a trait associated with an environment including a lifestyle.

For the identification of the association between the gene mutation and the trait, an exhaustive analysis of gene mutation using a next-generation sequencer, a microarray, or the like is usually utilized (Patent Literature 1). However, since a large number of gene mutations are found as candidates by the analysis, it is necessary to determine which gene mutation is associated with which trait and to select a gene mutation which is relatively highly associated with the trait.

### CITATION LIST

### Patent Literature

Patent Literature 1: JP 2018-191716 A

### SUMMARY OF INVENTION

### Technical Problem

While a large number of gene mutations are found as candidates as described above, the association between gene mutations in a gene mutation group is not clear. For this reason, in the current analysis, inferring the association between each mutation at a single position and the trait is the only way. However, when the association with a trait is analyzed focusing on only one locus mutation, for example, despite the mutation which actually affects the trait, there is a possibility that the association with the trait cannot be detected (false negative) due to the detection error of the mutation, measurement error of the trait, and the like and that such a mutation is missed as a gene mutation candidate having an association with the trait.

It is therefore an object of the present invention to provide a new gene mutation assessment system which makes it possible, even when a gene mutation has considered to be apparently not associated with a trait from mutation information at a single position, to pick the gene mutation as a gene mutation candidate showing an association with the trait, for example.

### Solution to Problem

In order to achieve the aforementioned object, the present invention provides a gene mutation assessment device, including: a communication unit; an assessment target mutation information acquisition unit; a score assignment unit; a score determination unit; a region mutation information acquisition unit; a score re-assignment unit; and an assessment score determination unit, wherein the communication unit can communicate with a database in which information on a gene mutation for a trait is stored, the assessment target mutation information acquisition unit acquires mutation information of a common gene mutation in a sample group showing a common trait as mutation information of an assessment target mutation, the mutation information includes position information of a mutation and base information of a mutation, the score assignment unit assigns a first score showing an association with a trait in the database information to the assessment target mutation based on the database information, the score determination unit compares the first score of the assessment target mutation with an association threshold and determines the assessment target mutation as a re-scoring target when the first score is less than the association threshold, the region mutation information acquisition unit acquires, as region mutation information, a gene mutation in an associated region with respect to a re-scoring target assessment target mutation based on the database information, the score re-assignment unit assigns a second score weighted to the first score to the re-scoring target assessment target mutation based on the region mutation information, and the assessment score determination unit determines the second score as an assessment score of the re-scoring target assessment target mutation.

The present invention also provides a gene mutation assessment method, including: an assessment target mutation information acquiring step; a score assigning step; a score determining step; a region mutation information acquiring step; a score re-assigning step; and an assessment score determining step, wherein the method is capable of communicating with a database in which information on a gene mutation for a trait is stored, the assessment target mutation information acquiring step acquires mutation information of a common gene mutation in a sample group showing a common trait as mutation information of an assessment target mutation, the mutation information includes position information of a mutation and base information of a mutation, the score assigning step assigns a first score showing an association with a trait in the database information to the assessment target mutation based on the database information, the score determining step compares the first score of the assessment target mutation with an association threshold and determines the assessment target mutation as a re-scoring target when the first score is less than the association threshold, the region mutation information acquiring step acquires, as region mutation information, a gene mutation in an associated region with respect to the re-scoring target assessment target mutation based on the database information, the score re-assigning step assigns a second score weighted to the first score to the re-scoring target assessment target mutation based on the region mutation information, and the assessment score determining step determines the second score as an assessment score of the re-scoring target assessment target mutation.

The present invention also provides a program for causing a computer to execute the gene mutation assessment method according to the present invention.

The present invention also provides a computer readable storage medium with the program according to the present invention.

### Advantageous Effects of Invention

According to the present invention, for example, even when it cannot be apparently determined that a gene mutation at a single position is associated with a trait, by referring to information on an associated region of the gene mutation, it is possible to pick a gene mutation having a possibility of showing an association with the trait. Therefore, the association between the gene mutation and the trait can be assessed more efficiently.

### BRIEF DESCRIPTION OF DRAWINGS

FIG. 1 is a block diagram showing an example of an assessment device according to the first example embodiment.

FIG. 2 is a block diagram showing an example of the hardware configuration of the assessment device according to the first example embodiment.

FIG. 3 is a flowchart showing an example of the assessment method according to the first example embodiment.

FIGS. 4A to 4C are simulation graphs showing the relationship between the degree of association with a trait and the chromosomal position.

FIG. 5 is a graph visualizing the relationship between the assessment target mutation and the assessment score showing the association with the trait in the second example embodiment.

### DESCRIPTION OF EMBODIMENTS

Example embodiments of the present invention will be described. Note here that the present invention is not limited to the following example embodiments. In the drawings, identical parts are denoted by identical reference numerals. Each example embodiment can be described with reference to the descriptions of other example embodiments, unless otherwise specified, and the configurations of the example embodiments may be combined, unless otherwise specified.

### First Example Embodiment

### (1) Assessment Device

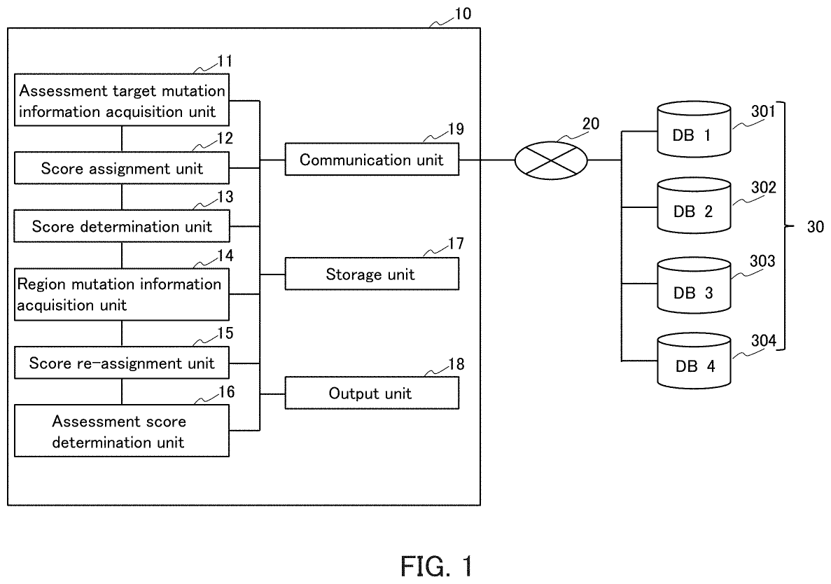

FIG. 1 is a block diagram showing the configuration of an example of a gene mutation assessment device 10 according to the present example embodiment. As shown in FIG. 1, the assessment device 10 includes an assessment target mutation information acquisition unit 11, a score assignment unit 12, a score determination unit 13, a region mutation information acquisition unit 14, a score re-assignment unit 15, an assessment score determination unit 16, and a communication unit 19. The assessment device 10 may further include, for example, a storage unit 17 and an output unit 18. The assessment device 10 is also referred to as an assessment system, for example.

The assessment device 10 may be a single assessment device including the respective units or may be an assessment device to which the respective units are connectable via a communication network, for example.

The assessment device 10 includes a communication unit 19 and is capable of communicating with a database 30 (301, 302, 303, 304). For example, as shown in FIG. 1, the assessment device 10 and the database 30 are capable of communicating with each other via a communication network 20 by a communication unit 19. The communication network 20 is not particularly limited, and a known network can be used, and may be, for example, a wired network or a wireless network. Examples of the communication network 20 include an internet line, a telephone line, a LAN (Local Area Network), and a WiFi (Wireless Fidelity). While the assessment device 10 and the database 30 are connected via the communication network 20 by the communication unit 19 in the first example embodiment as an example, the present invention is not limited thereto, and the assessment device 10 and the database 30 can be electrically connected by wire by the communication unit 19, thereby enabling communication, for example. The wired connection may be, for example, a cord connection or a cable connection for using a communication network.

The type and the number of the databases 30 communicating with the assessment device 10 are not limited, for example. The database 30 may be a database in which information on gene mutations for traits is stored. As the database 30, for example, a public database may be used, and examples thereof include PolyPhen, ExAC, Clinvar, Japanese genomic data (iJGVD), SIFT, and CADD. Further, in the present invention, the database is not limited to a database existing at the time of filing of the present application, for example, and a new database after filing can be used.

In the information of the database 30, the type of the trait is not particularly limited, and examples thereof include various traits such as diseases, responsiveness to drugs, traits associated with lifestyle, traits of physical characteristics, and traits such as exercise abilities or academic abilities. As the disease, for example, the classification of the International Disease Classification Table can be used. When the trait is a disease, for example, the gene mutation for the trait is a gene mutation that is significantly different between the group of patients with the disease and the group of normal individuals. When the trait is a specific disease, for example, the gene mutation for the trait is a gene mutation that is significantly different between the group of patients with the specific disease and the group of patients who are not infected with the specific disease (e.g., the group of normal individuals for the specific disease or the group of healthy individuals).

The assessment target mutation information acquisition unit 11 acquires mutation information of a common gene mutation in a sample group showing a common trait as mutation information of an assessment target mutation. The method for acquiring the mutation information is not particularly limited. The assessment target mutation information acquisition unit 11 may acquire the mutation information by input of a user using the input device to be described below, or may acquire the mutation information by reception from a database or the like via the communication network, for example.

The mutation information includes position information of the mutation and base information of the mutation. The position information is, for example, information on the position of the assessment target mutation in the gene, and the base information is, for example, information on the type of the base at the position in the gene. The format of the mutation information is not particularly limited, and may be, for example, a file format such as text data or a VCF file.

The sample group is a sample group showing a common trait. The type of the trait is not limited in any way as described above, and any trait can be set. Examples of the type of the trait include various traits such as diseases, responsiveness to drugs, traits associated with lifestyle, traits of physical characteristics, and traits such as exercise abilities or academic abilities. When the common trait of the sample group is a disease, the assessment target mutation is, for example, a gene mutation that is significantly different between the group of patients with the disease and the group of normal individuals. The common gene mutation may be acquired from, for example, information such as databases, papers, and the like, or may be extracted and acquired from mutation information on the sample group X⁺ showing the trait X and the mutation information on the sample group X⁻ not showing the trait X. The type of the sample group is not particularly limited, and examples of the sample group include sample groups classified by various factors such as presence or absence of disease, severity of disease, cohort, race, sex, age, and the like.

The number of common gene mutations in the sample group is not particularly limited, and may be, for example, one or two or more. For example, the assessment target mutation information acquisition unit 11 may acquire mutation information on a plurality of common gene mutations in the sample group.

The score assignment unit 12 assigns a first score showing the association with the trait in the database information to the assessment target mutation based on the database information. The score showing the association with the trait is preferably, for example, a relative value by which the magnitude of the association can be compared. As to the relative value, in the case where a score of 0 (zero) is set when no association is shown, and a score of 1 is set when the highest association is shown, a score closer to 0 can be given as the association is smaller, and a score closer to 1 can be given as the association is larger.

When the assessment device 10 can communicate with a plurality of databases by the communication unit 19, for example, the score assignment unit 12 may calculate the score of the assessment target mutation for each of the plurality of databases based on the database information, integrate the scores of the respective databases, and set the integrated score as the first score of the assessment target mutation. The method for calculating the integrated score is not particularly limited, and the integrated score can be calculated by a weighted linear sum using the scores of the respective databases, for example. The databases generally have different scales of values. For this reason, for example, by performing scoring based on the relative values and integrating the scores as described above, it is possible to avoid the influence due to the difference of values of the respective databases.

The score for each database may be weighted based on the accuracy of the database, for example. The accuracy of the database can be set as appropriate, for example.

The score determination unit 13 compares the first score of the assessment target mutation with the association threshold and determines the assessment target mutation as a re-scoring target when the first score is less than the association threshold. The threshold is not specifically limited and can be set as appropriate. For example, the score determination unit 13 may compare the first score of the assessment target mutation with the association threshold, and the assessment target mutation may be determined as a mutation associated with the trait in the database information if the first score satisfies the association threshold.

The region mutation information acquisition unit 14 acquires, as region mutation information, a gene mutation in an associated region with respect to the re-scoring target assessment target mutation based on the database information. The associated region is not particularly limited and can be set as appropriate. The information of the associated region with respect to the assessment target mutation may be stored in advance in the storage unit 17, for example.

The length of the associated region is not particularly limited, and can be set as appropriate, and as a specific example, the length is, for example, ±10,000 bases long, ±100,000 bases long, or the like. The associated region may be, for example, a contiguous sequence including the position of the assessment target mutation. The associated region may be, for example, a position of linkage with respect to the position of the assessment target mutation, a combination of positions of a plurality of linkages, or a region including the position of the linkage. The associated region may be, for example, a coding region, a structural domain, or the like associated with a gene having the assessment target mutation.

The score re-assignment unit 15 assigns a second score weighted to the first score to the re-scoring target assessment target mutation based on the region mutation information.

For example, the assessment score determination unit 16 determines the first score as the assessment score of the assessment target mutation when the first score of the assessment target mutation satisfies the threshold, and determines the second score as the assessment score of the re-scoring target assessment target mutation when the first score of the assessment target mutation does not satisfy the threshold.

In the assessment device 10, for example, the score determination unit 13 may also serve as an associated gene mutation determination unit. The associated gene mutation determination unit may compare the assessment score with the association threshold, and determine that the assessment target mutation whose assessment score satisfies the association threshold is a mutation associated with the trait in the database information.

When the assessment device 10 includes the storage unit 17, the storage unit 17 may store, for example, information from the database 30, information used for processing in each unit of the assessment device 10, and information obtained by processing in each unit of the assessment device 10. In the assessment device 10, the storage unit 17 may be the database 30.

When the assessment device 10 includes the output unit 18, the output unit 18 may output information obtained by processing in each unit of the assessment device 10, for example. The output destination by the output unit 18 may be a display when the assessment device 10 includes a display or the output destination by the output unit 18 may be external equipment to be described below, for example. In the latter case, the assessment device 10 and the external equipment are connectable via a communication network, for example.

(2) Hardware Configuration

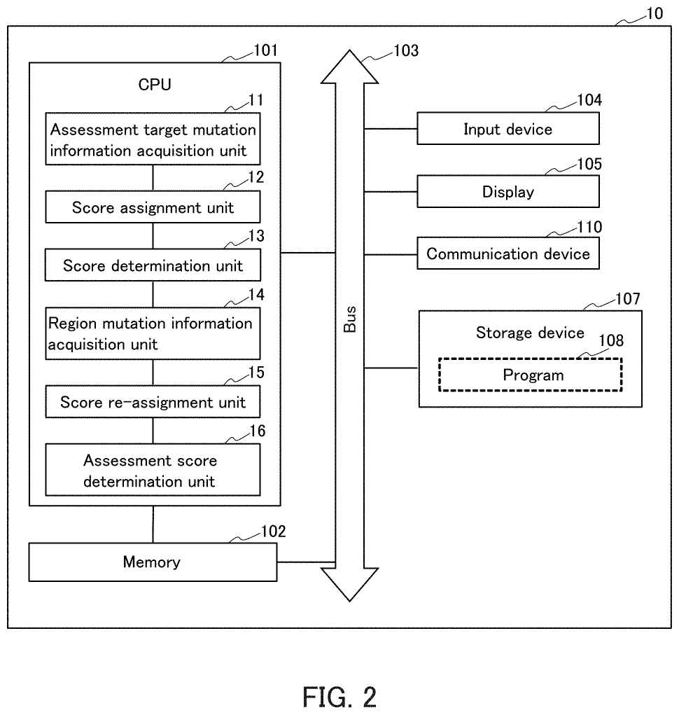

FIG. 2 illustrates a block diagram of the hardware configuration of the assessment device 10. The assessment device 10 includes, for example, a CPU (central processing unit) 101, a memory 102, a bus 103, an input device 104, a display 105, a communication device 110, a storage device 107, and the like. The respective units of the assessment device 10 are connected to each other via the bus 103 through respective interfaces (I/F) of the units, for example.

The CPU 101 is responsible for the entire control of the assessment device 10. In the assessment device 10, the CPU 101 executes the program of the present invention or other programs, and reads and writes various kinds of information, for example. Specifically, for example, the CPU 101 of the assessment device 10 functions as the assessment target mutation information acquisition unit 11, the score assignment unit 12, the score determination unit 13, the region mutation information acquisition unit 14, the score re-assignment unit 15, and the assessment score determination unit 16.

The bus 103 connects the respective functional units of the CPU 101, the memory 102, and the like, for example. The bus 103 can also be connected to external equipment, for example. The external equipment may be, for example, the database 30, a display terminal, or the like. The assessment device 10 can be connected to the communication network 20 by the communication device 110 connected to the bus 103, and can also be connected to the external equipment via the communication network 20. The communication device 110 is, for example, the communication unit 19.

The memory 102 includes, for example, a main memory, which is also referred to as a main memory. When the CPU 101 performs processing, the memory 102 reads various operation programs 108 such as the program of the present invention stored in the auxiliary storage device to be described below, and the CPU 101 receives data from the memory 102 and executes the program 108. The main memory is, for example, a RAM (random access memory). The memory 102 further includes, for example, a ROM (read-only memory).

The storage device 107 is also referred to as a so-called auxiliary storage in comparison with the main memory (main storage device), for example. The storage device 107 includes a storage medium and a drive for reading from and writing to the storage medium, for example. The storage medium is not particularly limited, and may be, for example, a built-in type or an external type, and examples thereof include HDs (hard disks), FDs (Floppy® disks), CD-ROMs, CD-Rs, CD-RWs, MOs, DVDs, flash memories, and memory cards. The drive is not particularly limited. The storage device 107 may be, for example, a hard disk drive (HDD) in which a storage medium and a drive are integrated. For example, as described above, the operation program 108 is stored in the storage device 107. Further, the storage device 107 may be the storage unit of the assessment device 10 and may store information input to the assessment device 10, information generated by the assessment device 10, or the like, for example.

The assessment device 10 further includes an input device 104, a display 105, and the like, for example. Examples of the input device 104 include a touch panel, a keyboard, and a mouse. Examples of the display 105 include an LED display and a liquid crystal display, and the display 105 serves as the output unit 18, for example.

(3) Gene Mutation Assessment Method

The assessment method of the present example embodiment can be performed using the assessment device 10 shown in FIGS. 1 and 2, for example. The assessment method of the present example embodiment is not limited to the use of the assessment device 10 shown in FIGS. 1 and 2. The description of the assessment method of the present example embodiment can be incorporated into the description of the assessment device 10 described above.

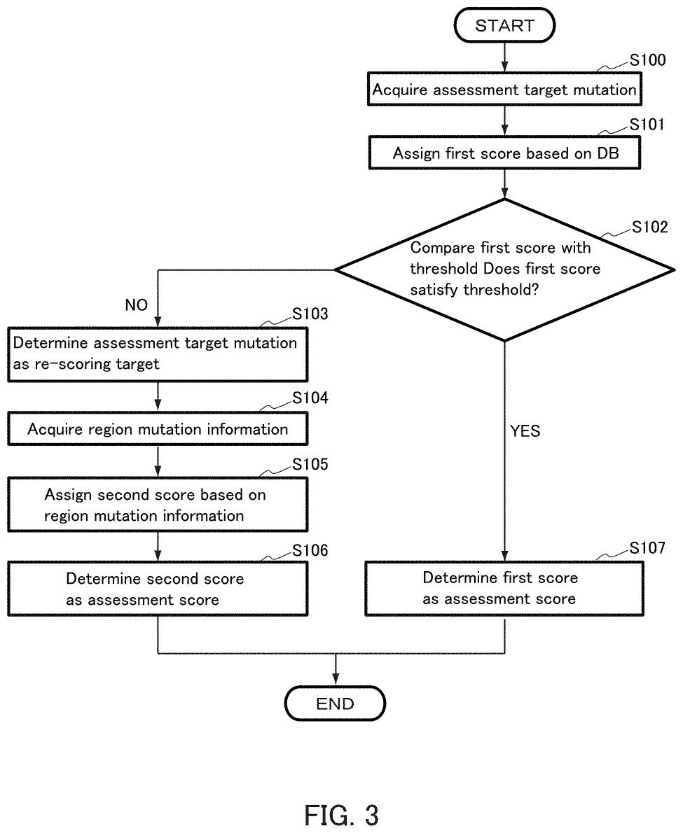

The assessment method of the present example embodiment will be described with reference to FIG. 3. FIG. 3 is a flowchart showing an example of the assessment method. In the following description, as an example, a case in which there are a plurality of common mutations in a sample group and the assessment is performed on these assessment target mutations based on one database information will be described. The plurality of assessment target mutations may be processed in parallel or sequentially, for example.

First, as an assessment target mutation information acquiring step, mutation information on a common gene mutation in a sample group showing a common trait is acquired as mutation information of the assessment target mutation (S100). This step can be performed by the assessment target mutation information acquisition unit 11 of the assessment device 10, for example.

The number (n) of common gene mutations in the sample group is not particularly limited, and may be one or two or more. In the present example embodiment, as a specific example, the following four types of gene mutations (mutations M1, M2, M3, and M4) are exemplified as common gene mutations in the sample group.

TABLE 1

| | Mutation | First | Threshold | Second | Assessment |
|---|---|---|---|---|---|
| Mutation | information | score | 0.5 | score | score |
| Mutation 1 | Chromosome 1 | 0.9 | Threshold or | — | 0.9 |
| (M 1) | 1,000th base | | more | | |
| Mutation 2 | Chromosome 3 | 0.1 | Less than | 0.8 | 0.8 |
| (M 2) | 12,500th base | | threshold | | |
| Mutation 3 | Chromosome 12 | 0.3 | Less than | 0.9 | 0.9 |
| (M 3) | 8,000th base | | threshold | | |
| Mutation 4 | Chromosome 19 | 0.1 | Less than | 0.6 | 0.6 |
| (M 4) | 470,000th base | | threshold | | |

Next, as the score assigning step, the first score showing the association with the trait in the database information is assigned to the assessment target mutation based on the database information (S101). This step can be performed, for example, by the scoring unit 12 of the assessment device 10.

In a specific example, Database 1 (DB1) in which gene mutation information for a trait A is stored is referred to, for example. The DB1 is considered to also contain information on the association between the trait A and each of the mutations M1 to M4. Then, when the first score showing the association between each of the mutations M1 to M4 and the trait A is assigned based on the information of the DB1, for example, the first scores, 0.9, 0.1, 0.3, and 0.1, can be assigned to the mutations M1 to M4, respectively, as shown in the Table 1. From this first score, it can be seen that the level of the association with respect to the trait A is in the order of the mutation M1, M3, M2, and M4.

Then, as the score determining step, the first score of the assessment target mutation is compared with the association threshold, and it is determined whether or not the first score satisfies the threshold (S102). When the first score is less than the association threshold (NO), the assessment target mutation is determined as a re-scoring target (S103). These steps can be performed by, for example, the score determination unit 13 of the assessment device 10.

The threshold can be set as appropriate as described above. In the case where the score is set to be larger as the association is higher and smaller as the association is lower, for example, when the first score is less than (or equal to or less than) the threshold, the assessment target mutation can be determined as a re-scoring target. On the other hand, in the case where the score is set to be smaller as the association is higher and larger as the association is lower, for example, when the first score exceeds the threshold (or is equal to or larger than the threshold), the assessment target mutation can be determined as a re-scoring target.

In the usual method, as to the assessment target mutation, when the first score showing the association with the trait is less than the threshold, which is a criterion, the assessment target mutation is excluded as being unassociated with the trait. However, some of such assessment target mutations may actually be associated with the trait. In contrast, the present invention makes it possible to pick the assessment target mutation having the possibility of being actually associated with the trait by assigning a further score to the assessment target mutation having the first score of less than the threshold, as described below.

In the specific example, for example, when the threshold is 0.5, the first scores of the mutation M2, M3, and M4 are less than the threshold as shown in the Table 1, and therefore, the assessment target mutations are determined as a re-scoring target.

Next, as the region mutation information acquiring step, a gene mutation in an associated region with respect to the re-scoring target assessment target mutation is acquired as region mutation information based on the database information (S104). This step can be performed, for example, by the region mutation information acquisition unit 14 of the assessment device 10. Then, as the score re-assigning step, a second score weighted to the first score is assigned to the re-scoring target assessment target mutation based on the region mutation information (S105). This step can be performed, for example, by the score re-assignment unit 15 of the assessment device 10.

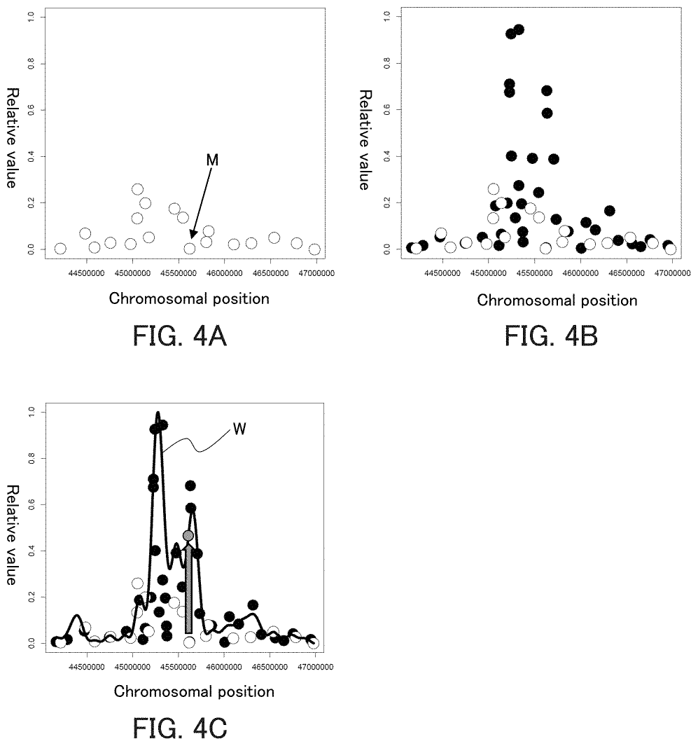

These steps are based on the findings obtained by the inventors of the present invention. Hence, the findings obtained by the inventors of the present invention will be described with reference to the simulation graphs of FIGS. 4A to 4C. FIGS. 4A to 4C are simulation graphs for explaining the present example embodiment, and the chromosomal position, the numerical value of the relative value, and the like are merely examples. In addition, the present invention is not limited to the following description.

FIG. 4A is a simulation graph showing the relative values with respect to the trait A as to a plurality of assessment target mutations detected from sequences of a sample group, the X-axis indicates the chromosomal position, and the Y-axis indicates the relative value (white circle) with respect to the trait A shown by a database. The relative value means the degree of influence (also referred to as degree of harm or association) of the mutation on the trait as described above. In FIGS. 4A to 4C, the relative value is shown in the range where the lower limit is 0 and the upper limit is 1. However, the relative value is not limited thereto, and may be, for example, a value shown in each database. Specifically, for example, in the association analysis, the relative value can also be represented by −log 10 p values. In FIG. 4A, the assessment target mutation M at the chromosomal position identified by the arrow shows only a very low relative value with respect to the trait A. Thus, when only a single position is considered, this mutation M is eliminated as unassociated with the trait A.

Next, FIG. 4B is a graph obtained by plotting, as to mutations that could not be detected or were not detected in the sequence of the sample group, the relative values with respect to the traits registered in the database on the same simulation graph as in FIG. 4A (black circles). As shown in FIG. 4B, mutations showing extremely high relative values with respect to the trait are clustered around the mutation M. As to the gene mutation, generally, the mutation itself may directly affect the trait, or the mutation itself may not directly affect the trait but the mutation around or in linkage with the mutation may affect the trait. For this reason, even when the relative value is determined to be low by the first score, by referring to the mutation information in the associated region of the mutation M, it is conceivable that the mutation M may actually show an association with the trait A.

As shown in FIG. 4C, for example, a density curve (W) of the mutation is generated from plots (black circles) of the mutation information around the mutation M. By weighting the relative value of the mutation M based on this density curve, the relative value of the mutation M can be raised to the relative value on the density curve, as indicated by the arrow. The density curve (W) can be obtained, for example, by interpolation using a kernel function. In addition to the method using the kernel function, for example, the second score may be assigned by weighting according to the distance on the chromosome. That is, by utilizing the region mutation information of the associated region of the assessment target mutation M in this manner, it is also possible to further asses a mutation that is considered to be unassociated from the first score by assigning a weighted second score.

The associated region can be set as appropriate. The setting condition of the associated region may be stored in advance in the storage unit 17, for example. In this case, in the case where the associated region is a contiguous sequence including the assessment target mutation as described above, for example, the position of the assessment target mutation in the contiguous sequence, the length of the contiguous sequence, and the like can be set as the setting condition. When the associated region is a position of a linkage with respect to the position of the assessment target mutation as described above, for example, the position of the linkage with respect to the position for each mutation can be set as the setting condition. The region mutation information in the associated region can be obtained from the database information.

In a specific example, associated regions are set for the re-scoring target mutations M2, M3, and M4, respectively, and the gene mutation in each associated region is acquired as region mutation information. The gene mutation in the associated region may be, for example, a gene mutation for the trait A or a gene mutation for other traits. That is, for example, the relative values of the gene mutations of the sample group with respect to the trait A (breast cancer) may be plotted with white circles in FIG. 4A, and further, the relative values of the gene mutations at various chromosomal positions registered in the database with respect to the breast cancer may be plotted with black circles in FIG. 4B. Also, for example, the relative values of the gene mutations of the sample group with respect to the trait A (breast cancer) may be plotted with white circles in FIG. 4A, and the relative values of the gene mutations at various chromosomal positions registered in the database with respect to other trait B (e.g., stomach cancer) may be plotted with black circles in FIG. 4B. Then, as shown in Table 1, the first score (0.1) of the mutation M2 is weighted to make the second score (0.8), the first score (0.3) of the mutation M3 is weighted to make the second score (0.9), and the first score (0.1) of the mutation M4 is weighted to make the second score (0.6) based on the respective region mutation information.

Then, the assessment score determining step determines the second score as an assessment score of the re-scoring target assessment target mutation (S106). These steps can be performed by the assessment score determination unit 16 of the assessment device 10, for example.

When it is determined in the step (S102) that the first score satisfies the association threshold (Yes), the first score is determined as an assessment score of the assessment target mutation (S107). These steps can be performed by the assessment score determination unit 16 of the assessment device 10, for example.

While the relative values of mutations that could not be detected in the sequence of the sample group with respect to the trait were plotted (black circles) to generate the density curve (W) in FIG. 4B, the present invention is not limited thereto. The relative value of the mutation detected in the sample group sequence shown in FIG. 4A with respect to the trait registered in the database may be further plotted to generate a density curve (W), and the second score of the mutation M may be assigned. In this case, since FIG. 4A shows the relative value with respect to the trait A, the relative values with respect to other trait B are plotted to the same mutation to generate the density curve (W), and the second score of the mutation M is assigned.

(Variation 1)

When the assessment device 10 is capable of communicating with a plurality of databases by the communication unit 19 as shown in FIG. 1, the score assignment unit 12 may calculate the score of the assessment target mutation for each of the plurality of databases based on the database information, integrate the scores of the respective databases, and set the integrated score as the first score of the assessment target mutation.

The integrated score is not particularly limited, and can be calculated by a weighted linear sum using the scores of the respective databases, for example. As for the weighted linear sum, statistical means such as, for example, a generalized linear model, a neural network, or the like can be utilized. The score assignment unit 12 may weight the score for each database based on the accuracy of the database.

As a specific example, as shown in Table 2 below, there are four types of gene mutations (mutations M1, M2, M3, and M4) as common gene mutations in the sample group, and four types of databases (DB1, DB2, DB3, DB4) are used.

TABLE 2

| | Score | | | | Integrated |
|---|---|---|---|---|---|
| Mutation | DB 1 | DB2 | DB3 | DB4 | score |
| Mutation 1 | 0.9 | 0.8 | 0.9 | 0.9 | 0.9 |
| (M 1) | | | | | |
| Mutation 2 | 0.2 | 0.1 | 0.2 | 0.3 | 0.1 |
| (M 2) | | | | | |
| Mutation 3 | 0.5 | 0.1 | 0.9 | 0.7 | 0.3 |
| (M 3) | | | | | |
| Mutation 4 | 0.1 | 0.2 | 0.1 | 0.1 | 0.1 |
| (M 4) | | | | | |

For each of the assessment mutations (M1, M2, M3, and M4), the score can be calculated based on each database information, and the integrated score can be obtained by the following model equation using the scores of the four types of databases. For the calculation of the integrated score, for example, machine learning such as unsupervised learning or supervised learning can be utilized. The unsupervised learning may be, for example, principal component analysis, and the supervised learning may be, for example, a support vector machine, a Naive Bayes classifier, or the like.

$$S c o r {e}_{i} = {β}_{0} + {\sum }_{j = 1}^{n} {β}_{i , j} {S}_{i , j}$$

i: i-th gene mutation
j: j-th database
n: Number of databases
β₀: Constant term representing intercept
S_{i,j}: Score for gene mutation i in database j
β_{i,j}: Weight of score for gene mutation i in database j

### Second Example Embodiment

The assessment device of the present example embodiment can further output the assessment score, for example. The output of the assessment score may include, for example, visualization data based on the assessment score.

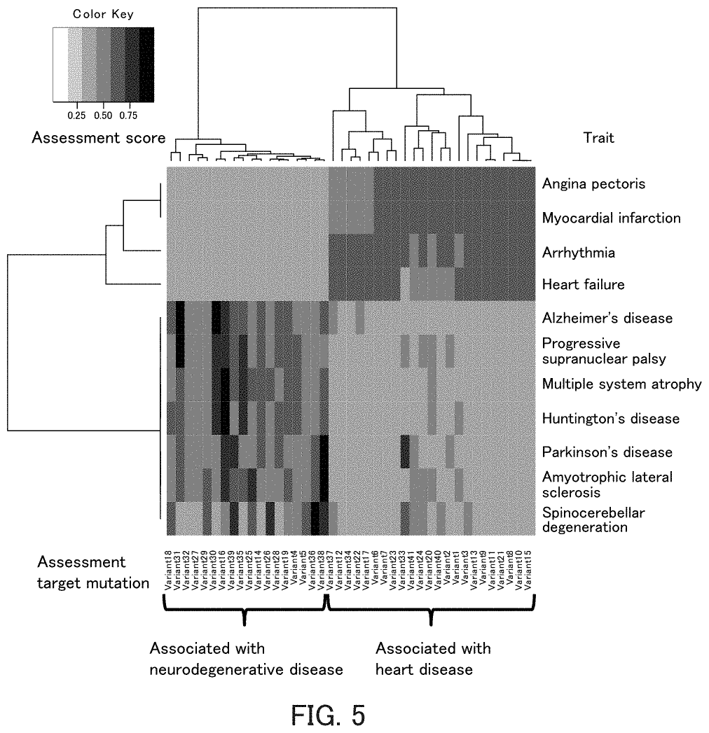

FIG. 5 is a graph of a numerical matrix showing the relationship between a plurality of assessment target mutations and an assessment score for each trait. In FIG. 5, the assessment target mutations are arranged in the row direction, and the disease traits are shown in the column direction. The higher the assessment score is, the darker the color is, and the lower the assessment score is, the lighter the color is. Specifically, in FIG. 5, the assessment score for the neurodegenerative disease and the assessment score for the cardiac disease are clustered.

As shown in FIG. 5, the assessment target mutation group on the left shows a high assessment score for the neurodegenerative disease, suggesting an association with the neurodegenerative disease. On the other hand, the assessment target mutation group on the right shows a high assessment score for the heart disease, suggesting an association with the heart disease. Note that the notation in FIG. 5 is not limited, and for example, the left group has a relatively high assessment score showing an association with the neurodegenerative disease, and the right group has a relatively high assessment score showing an association with the heart disease. As to the diseases indicated by the vertical axis, the upper group is the heart disease, and the upper group is the neurodegenerative disease.

As can be seen from the graph of FIG. 5, according to the present invention, since the association can be visualized by utilizing the relative assessment score, for example, it is possible to visually judge the relationship between a certain gene mutation and a certain trait, the relationship between a certain trait and a plurality of gene mutations, the relationship between a certain gene mutation and a plurality of traits, or the like, without being influenced by a huge numerical comparison or a scale different from one database to another.

In the present example embodiment, for the profile of the assessment target mutation and the disease, for example, hierarchical clustering, k-means method, and the like can also be used.

The format of the visualization data is not particularly limited, and may be the format of a numerical matrix as described above, or may be a bar graph, a plot graph, or the like.

### Third Example Embodiment

The program of the present example embodiment is a program capable of causing a computer to execute the assessment method of the present invention. Alternatively, the program of the present example embodiment may be recorded on, for example, a computer readable storage medium. The storage medium is not particularly limited, and may be, for example, a storage medium as described above, or the like.

While the present invention has been described above with reference to illustrative example embodiments, the present invention is by no means limited thereto. Various changes and variations that may become apparent to those skilled in the art may be made in the configuration and specifics of the present invention without departing from the scope of the present invention.

This application claims priority from Japanese Patent Application No. 2018-051268 filed on Mar. 19, 2018. The entire subject matter of the Japanese Patent Application is incorporated herein by reference.

(Supplementary Notes)

Some or all of the above example embodiments and examples may be described as in the following Supplementary Notes, but are not limited thereto.

### (Supplementary Note 1)

A gene mutation assessment device, including:

a communication unit;

an assessment target mutation information acquisition unit;

a score assignment unit;

a score determination unit;

a region mutation information acquisition unit;

a score re-assignment unit; and

an assessment score determination unit, wherein

the communication unit can communicate with a database in which information on a gene mutation for a trait is stored,

the assessment target mutation information acquisition unit acquires mutation information of a common gene mutation in a sample group showing a common trait as mutation information of an assessment target mutation,

the mutation information includes position information of a mutation and base information of a mutation,

the score assignment unit assigns a first score showing an association with a trait in the database information to the assessment target mutation based on the database information,

the score determination unit compares the first score of the assessment target mutation with an association threshold and determines the assessment target mutation as a re-scoring target when the first score is less than the association threshold,

the region mutation information acquisition unit acquires, as region mutation information, a gene mutation in an associated region with respect to a re-scoring target assessment target mutation based on the database information,

the score re-assignment unit assigns a second score weighted to the first score to the re-scoring target assessment target mutation based on the region mutation information, and

the assessment score determination unit determines the second score as an assessment score of the re-scoring target assessment target mutation.

### (Supplementary Note 2)

The assessment device according to Supplementary Note 1, wherein

the assessment score determination unit determines the first score as an assessment score of the assessment target mutation when the first score of the assessment target mutation satisfies the threshold, and determines the second score as the assessment score of the re-scoring target assessment target mutation when the first score of the assessment target mutation does not satisfy the threshold.

### (Supplementary Note 3)

The assessment device according to Supplementary Note 1 or 2, wherein

in the assessment target mutation information acquisition unit, the common trait of the sample group is a disease, and the assessment target mutation is a gene mutation that is significantly different between a group of patients with the disease and a group of normal individuals.

### (Supplementary Note 4)

The assessment device according to any one of Supplementary Notes 1 to 3, wherein

the assessment target mutation information acquisition unit acquires mutation information on a plurality of common gene mutations in the sample group.

### (Supplementary Note 5)

The assessment device according to any one of Supplementary Notes 1 to 4, wherein

the trait in the database information is a disease, and the gene mutation for the trait is a gene mutation that is significantly different between a group of patients with the disease and a group of normal individuals.

### (Supplementary Note 6)

The assessment device according to any one of Supplementary Notes 1 to 5, wherein

the trait in the database information is a specific disease, and the gene mutation for the trait is a gene mutation that is significantly different between a group of patients with the specific disease and a group of normal individuals.

### (Supplementary Note 7)

The assessment device according to any one of Supplementary Notes 1 to 6, wherein

in the region mutation information acquisition unit, the associated region is a contiguous sequence including a position of the assessment target mutation.

### (Supplementary Note 8)

The assessment device according to any one of Supplementary Notes 1 to 6, wherein

in the region mutation information acquisition unit, the associated region includes a position of a linkage with respect to a position of the assessment target mutation.

### (Supplementary Note 9)

The assessment device according to any one of Supplementary Notes 1 to 8, wherein

the communication unit can communicate with a plurality of databases, and the score assignment unit calculates a score of the assessment target mutation for each of the plurality of databases based on the database information, integrates the scores of the respective databases, and sets the integrated score as the first score of the assessment target mutation.

### (Supplementary Note 10)

The assessment device according to Supplementary Note 9, wherein

the score assignment unit calculates the integrated score by a weighted linear sum using the scores of the respective databases.

### (Supplementary Note 11)

The assessment device according to Supplementary Note 9 or 10, wherein

the score assignment unit weights the score for each database based on an accuracy of the database.

### (Supplementary Note 12)

The assessment device according to any one of Supplementary Notes 1 to 11, wherein

the score assignment unit assigns a relatively large score as an association with the trait is relatively high, and assigns a relatively small score as the association with the trait is relatively low.

### (Supplementary Note 13)

The assessment device according to any one of Supplementary Notes 1 to 12, wherein

the score determination unit compares the assessment score with the association threshold, and determines an assessment target mutation whose assessment score satisfies the association threshold as a mutation associated with the trait in the database information.

### (Supplementary Note 14)

The assessment device according to any one of Supplementary Notes 1 to 13, further including:

a storage unit, wherein

the storage unit stores the assessment score in association with each assessment target mutation.

### (Supplementary Note 15)

The assessment device according to any one of Supplementary Notes 1 to 14, further including:

an output unit, wherein

the output unit outputs an assessment score showing an association with the trait in association with each assessment target mutation.

### (Supplementary Note 16)

The assessment device according to any one of Supplementary Notes 1 to 15, further including:

a storage unit, wherein

the storage unit stores the assessment score of the assessment target mutation in association with each trait in the database information.

### (Supplementary Note 17)

The assessment device according to any one of Supplementary Notes 1 to 16, further including:

an output unit, wherein

the output unit outputs the assessment score of the assessment target mutation in association with each trait in the database information.

### (Supplementary Note 18)

The assessment device according to Supplementary Note 15 or 17, wherein

the output unit outputs the assessment score as visualization data.

### (Supplementary Note 19)

A gene mutation assessment method, including:

an assessment target mutation information acquiring step;

a score assigning step;

a score determining step;

a region mutation information acquiring step;

a score re-assigning step; and

an assessment score determining step, wherein

the method is capable of communicating with a database in which information on a gene mutation for a trait is stored,

the assessment target mutation information acquiring step acquires mutation information of a common gene mutation in a sample group showing a common trait as mutation information of an assessment target mutation,

the mutation information includes position information of a mutation and base information of a mutation,

the score assigning step assigns a first score showing an association with a trait in the database information to the assessment target mutation based on the database information,

the score determining step compares the first score of the assessment target mutation with an association threshold and determines the assessment target mutation as a re-scoring target when the first score is less than the association threshold,

the region mutation information acquiring step acquires, as region mutation information, a gene mutation in an associated region with respect to the re-scoring target assessment target mutation based on the database information,

the score re-assigning step assigns a second score weighted to the first score to the re-scoring target assessment target mutation based on the region mutation information, and

the assessment score determining step determines the second score as an assessment score of the re-scoring target assessment target mutation.

### (Supplementary Note 20)

The assessment method according to Supplementary Note 19, wherein

the assessment score determining step determines the first score as an assessment score of the assessment target mutation when the first score of the assessment target mutation satisfies the threshold, and determines the second score as the assessment score of the re-scoring target assessment target mutation when the first score of the assessment target mutation does not satisfy the threshold.

### (Supplementary Note 21)

The assessment method according to Supplementary Note 19 or 20, wherein

in the assessment target mutation information acquiring step, the common trait of the sample group is a disease, and the assessment target mutation is a gene mutation that is significantly different between a group of patients with the disease and a group of normal individuals.

### (Supplementary Note 22)

The assessment method according to any one of Supplementary Notes 19 to 21, wherein

the assessment target mutation information acquiring step acquires mutation information on a plurality of common gene mutations in the sample group.

### (Supplementary Note 23)

The assessment method according to any one of Supplementary Notes 19 to 22, wherein

the trait in the database information is a disease, and the gene mutation for the trait is a gene mutation that is significantly different between a group of patients with the disease and a group of normal individuals.

### (Supplementary Note 24)

The assessment method according to any one of Supplementary Notes 19 to 23, wherein

the trait in the database information is a specific disease, and the gene mutation for the trait is a gene mutation that is significantly different between a group of patients with the specific disease and a group of normal individuals.

### (Supplementary Note 25)

The assessment method according to any one of Supplementary Notes 19 to 24, wherein

in the region mutation information acquiring step, the associated region is a contiguous sequence including a position of the assessment target mutation.

### (Supplementary Note 26)

The assessment method according to any one of Supplementary Notes 19 to 25, wherein

in the region mutation information acquiring step, the associated region includes a position of a linkage with respect to a position of the assessment target mutation.

### (Supplementary Note 27)

The assessment method according to any one of Supplementary Notes 19 to 26, wherein

the method is capable of communicating with a plurality of databases, and

the score assigning step calculates a score of the assessment target mutation for each of the plurality of databases based on the database information, integrates the scores of the respective databases, and sets the integrated score as the first score of the assessment target mutation.

### (Supplementary Note 28)

The assessment method according to Supplementary Note 27, wherein

the score assigning step calculates the integrated score by a weighted linear sum using the scores of the respective databases.

### (Supplementary Note 29)

The assessment method according to Supplementary Note 27 or 28, wherein

the score assigning step weights the score for each database based on an accuracy of the database.

### (Supplementary Note 30)

The assessment method according to any one of Supplementary Notes 19 to 29, wherein

the score assigning step assigns a relatively large score as an association with the trait is relatively high, and assigns a relatively small score as the association with the trait is relatively low.

### (Supplementary Note 31)

The assessment method according to any one of Supplementary Notes 19 to 30, wherein

the score determining step compares the assessment score with the association threshold, and determines an assessment target mutation whose assessment score satisfies the association threshold as a mutation associated with the trait in the database information.

### (Supplementary Note 32)

The assessment method according to any one of Supplementary Notes 19 to 31, further including:

a storing step, wherein

the storing step stores the assessment score in association with each assessment target mutation.

### (Supplementary Note 33)

The assessment method according to any one of Supplementary Notes 19 to 32, further including:

an outputting step, wherein

the outputting step outputs an assessment score showing an association with the trait in association with each assessment target mutation.

### (Supplementary Note 34)

The assessment method according to any one of Supplementary Notes 19 to 33, further including:

a storing step, wherein

the storing step stores the assessment score of the assessment target mutation in association with each trait in the database information.

### (Supplementary Note 35)

The assessment method according to any one of Supplementary Notes 19 to 34, further including:

an outputting step, wherein

the outputting step outputs the assessment score of the assessment target mutation in association with each trait in the database information.

### (Supplementary Note 36)

The assessment method according to Supplementary Note 33 or 35, wherein

the outputting step outputs the assessment score as visualization data.

### (Supplementary Note 37)

A program for causing a computer to execute the assessment method according to any one of Supplementary Notes 19 to 36.

### (Supplementary Note 38)

A computer readable storage medium with the program according to Supplementary Note 37.

### INDUSTRIAL APPLICABILITY

According to the present invention, for example, even when it cannot be apparently determined that a gene mutation at a single position is associated with a trait, by referring to information on an associated region of the gene mutation, it is possible to pick a gene mutation having a possibility of showing an association with the trait. Therefore, the association between the gene mutation and the trait can be assessed more efficiently.

### REFERENCE SIGNS LIST

10: Assessment device
11: Assessment target mutation information acquisition unit
12: Score assignment unit
13: Score determination unit
14: Region mutation information acquisition unit
15: Score re-assignment unit
16: Assessment score determination unit
17: Storage unit
18: Output unit
19: Communication unit
101: CPU
102: Memory
103: Bus
104: Input device
105: Display
107: Storage device
108: Program
110: Communication device
20: Communication Network
30: Database

1. A device for a gene mutation assessment, comprising at least one processor configured to:
be capable to communicate with a database in which information on a gene mutation for a trait is stored;
acquire mutation information of a common gene mutation in a sample group showing a common trait as mutation information of an assessment target mutation, wherein
the mutation information includes position information of a mutation and base information of a mutational;
assign a first score showing an association with a trait in the database information to the assessment target mutation based on the database informational;
compare the first score of the assessment target mutation with an association threshold;
determine the assessment target mutation as a re-scoring target when the first score is less than the association threshold;
acquire, as region mutation information, a gene mutation in an associated region with respect to a re-scoring target assessment target mutation based on the database information;
re-assign a second score weighted to the first score to the re-scoring target assessment target mutation based on the region mutation information; and
determine the second score as an assessment score of the re-scoring target assessment target mutation.

2. The device according to claim 1, wherein
the processor is configured to:
determine the first score as an assessment score of the assessment target mutation when the first score of the assessment target mutation satisfies the threshold; and
determine the second score as the assessment score of the re-scoring target assessment target mutation when the first score of the assessment target mutation does not satisfy the threshold.

3. The device according to claim 1, wherein
the common trait of the sample group is a disease, and the assessment target mutation is a gene mutation that is significantly different between a group of patients with the disease and a group of normal individuals.

4. The device according to claim 1, wherein
the processor is configured to acquire mutation information on a plurality of common gene mutations in the sample group.

5. The device according to claim 1, wherein
the trait in the database information is a disease, and the gene mutation for the trait is a gene mutation that is significantly different between a group of patients with the disease and a group of normal individuals.

6. The device according to claim 1, wherein
the trait in the database information is a specific disease, and the gene mutation for the trait is a gene mutation that is significantly different between a group of patients with the specific disease and a group of normal individuals.

7. The device according to claim 1, wherein
the associated region is a contiguous sequence including a position of the assessment target mutation.

8. The device according to claim 1, wherein
the associated region includes a position of a linkage with respect to a position of the assessment target mutation.

9. The device according to claim 1, wherein
the processor is configured to:
be capable to communicate with a plurality of databases, and
calculate a score of the assessment target mutation for each of the plurality of databases based on the database information;
integrate the scores of the respective databases; and
set the integrated score as the first score of the assessment target mutation.

10. The device according to claim 9, wherein
the processor is configured to calculate the integrated score by a weighted linear sum using the scores of the respective databases.

11. The device according to claim 9, wherein
the processor is configured to weight the score for each database based on an accuracy of the database.

12. The device according to claim 1, wherein
the processor is configured to:
assign a relatively large score as an association with the trait is relatively high; and
assign a relatively small score as the association with the trait is relatively low.

13. The device according to claim 1, wherein
the processor is configured to:
compare the assessment score with the association threshold; and
determine an assessment target mutation whose assessment score satisfies the association threshold as a mutation associated with the trait in the database information.

14. The device according to claim 1, further comprising:
a storage, wherein
the storage stores the assessment score in association with each assessment target mutation.

15. The device according to claim 1, wherein
the processor is configured to output an assessment score showing an association with the trait in association with each assessment target mutation.

16. The device according to claim 1, further comprising:
a storage, wherein
the storage stores the assessment score of the assessment target mutation in association with each trait in the database information.

17. The device according to claim 1, wherein
the processor is configured to output the assessment score of the assessment target mutation in association with each trait in the database information.

18. The device according to claim 15, wherein
the processor is configured to output the assessment score as visualization data.

19. A computer-implemented method for a gene mutation assessment, wherein
the computer is capable of communicating with a database in which information on a gene mutation for a trait is stored,
the method comprising:
acquiring mutation information of a common gene mutation in a sample group showing a common trait as mutation information of an assessment target mutation, wherein
the mutation information includes position information of a mutation and base information of a mutation;
assigning a first score showing an association with a trait in the database information to the assessment target mutation based on the database information,
comparing the first score of the assessment target mutation with an association threshold;
determining the assessment target mutation as a re-scoring target when the first score is less than the association threshold;
acquiring, as region mutation information, a gene mutation in an associated region with respect to the re-scoring target assessment target mutation based on the database information;
re-assigning a second score weighted to the first score to the re-scoring target assessment target mutation based on the region mutation information; and
determining the second score as an assessment score of the re-scoring target assessment target mutation.

20-37. (canceled)

38. A non-transitory computer readable storage medium with the program, wherein
the program cause a computer to execute a method for a gene mutation assessment, wherein,
the computer is capable of communicating with a database in which information on a gene mutation for a trait is stored, wherein
the method comprise:
acquiring mutation information of a common gene mutation in a sample group showing a common trait as mutation information of an assessment target mutation, wherein
the mutation information comprises position information of a mutation and base information of a mutation;
assigning a first score showing an association with a trait in the database information to the assessment target mutation based on the database information;
comparing the first score of the assessment target mutation with an association threshold;
determining the assessment target mutation as a re-scoring target when the first score is less than the association threshold;
acquiring, as region mutation information, a gene mutation in an associated region with respect to the re-scoring target assessment target mutation based on the database information,
re-assigning a second score weighted to the first score to the re-scoring target assessment target mutation based on the region mutation information; and
determining the second score as an assessment score of the re-scoring target assessment target mutation.
